(19) United States
(12) Plant Patent Application Publication
(10) Pub. No.: US 2003/0208808 P1
(43) Pub. Date: Nov. 6, 2003

(54) Prunus L. variety named 'Piku 4'

(76) Inventor: Brigitte Wolfram, Dresden (DE)

Correspondence Address: Russell D. Orkin, ORKIN & HANSON, P.C., 700 Koppers Building, 436 Seventh Avenue, Pittsburgh, PA, 15219-1818, US

(21) Appl. No.: 10/136,467

(22) Filed: May 1, 2002

(51) Int. Cl. A01H 5/00

(52) U.S. Cl. PLT/183

(57) ABSTRACT

A new and distinct Prunus rootstock compatible with other Prunus varieties and producing trees with reduced growth, suitable to sandy soil with high productivity.

### CLASSIFICATION

Prunus L.

### VARIETAL DENOMINATION

‘Piku 4’

### BACKGROUND OF THE INVENTION

The present invention comprises a new and distinct cultivar of hybrid Prunus known by the varietal name ‘Piku 4’. The new variety was discovered in 1976 in a selective breeding program designed to produce growth reducing rootstock for cherry trees in Dresden-Pilluitz, Germany as a cross between an unnamed, unpatented variety of Prunus cerasus (female parent) and an unnamed, unpatented variety of Prunus kunsar (P. kansuensis×P. sargentii) (male parent). The new variety differs from its parents in its growth habit. The following characteristic distinguish the new variety from Prunus avium varieties:

1. Three-way hybrid.

2. Reduces growth in grafted varieties by 30-40%.

3. Suitable for sandy soil.

4. Precocious crop.

5. Glossiness of upper leaf surface.

6. Growth habit.

The new variety was first asexually reproduced via green cuttings in 1980 in Dresden-Pilluitz, Germany and subsequent propagation therein has confirmed that the characteristics of the new variety are stable and are transmitted to subsequent generations.

### DESCRIPTION OF THE DRAWING

The accompanying photographic drawings illustrate the new variety, with the color being as nearly true as is possible with color illustrations of this type.

FIG. 1 shows the tree of the new variety;

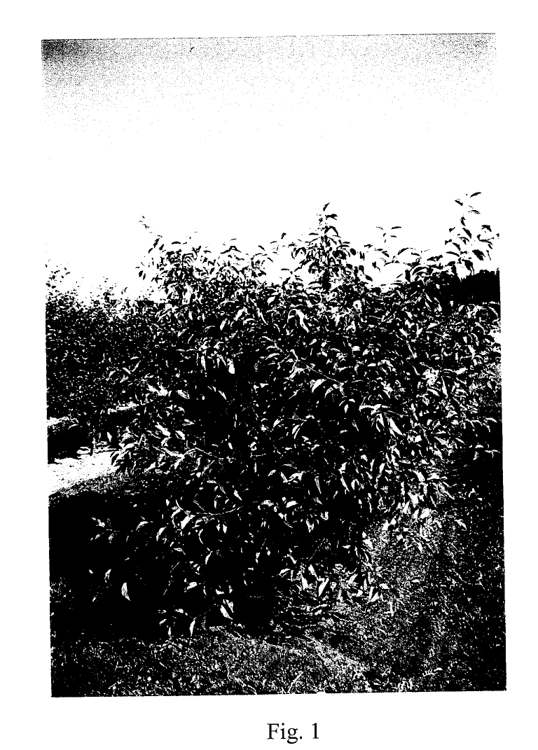

FIG. 2 shows a branch with leaves; and

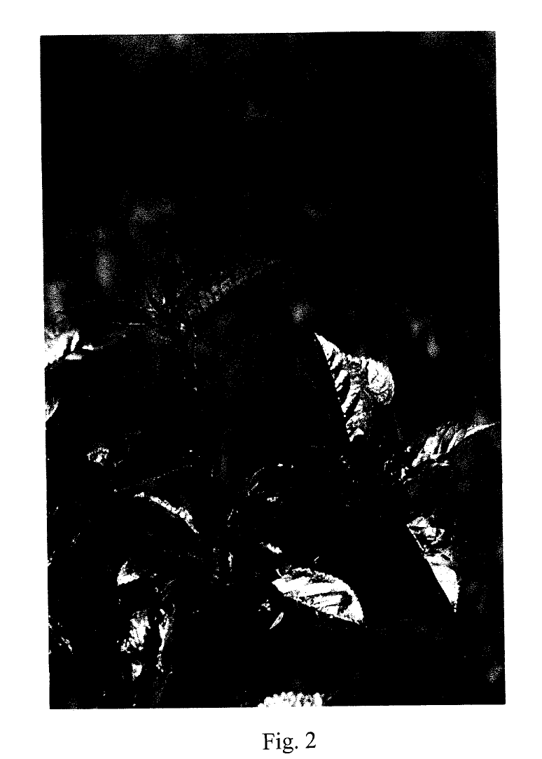

FIG. 3 shows a branch with flower buds;

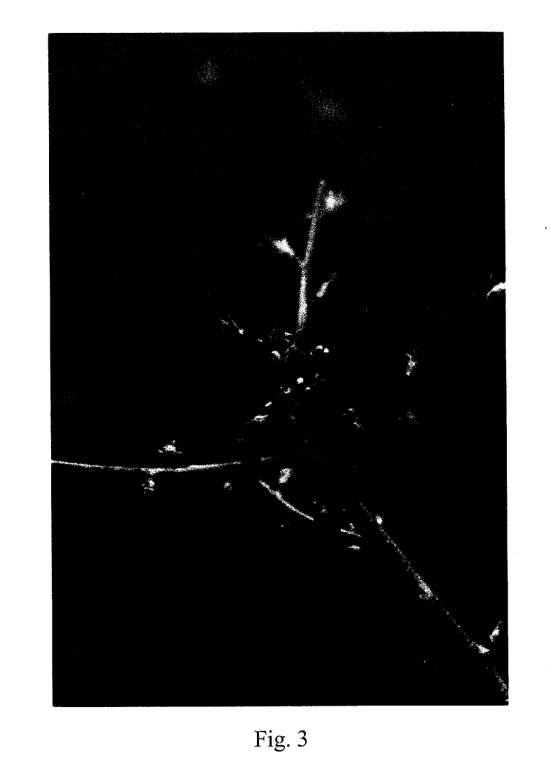

### DESCRIPTION OF THE PLANT

The following detailed description sets forth the characteristics of the new cultivar. The data which defines these characteristics were collected by asexual reproductions via green cuttings carried out in Dresden-Pilluitz, Germany. The color measurements were taken in outdoors Dresden-Pilluitz, Germany. Color designations are noted with reference to The Royal Horticulture Society Colour Chart. The new variety prefers dry or sandy soil and is resistant to PNRV and PDV. As a rootstock, it can be reproduced via softwood cuttings. The new variety anchors adequately with some root sprouts, sufficient compatability with grafts and exhibits dwarf growth, vigor.

### TREE

Size: Small-medium.

Form: Bushy, drooping.

Trunk:

Size.—Small.

Surface texture.—Smooth.

Bark color.—Dark.

Branches:

Surface texture.—Smooth.

Bark color.—Dark brown.

New growth color.—Light red.

Mature growth color.—Brown.

Internode length.—Medium.

Bud.—Medium, round.

Leaves:

Length.—10.4 cm.

Width.—5.0 cm.

Surface texture.—Upper side glossy.

Form.—Elongated, elliptical.

Color.—Light green.

Petioles:

Length.—1.7 cm.

Thickness.—Thin-medium.

Leaf glands:

Quantity.—1-4.

Position.—Under leaf end.

Size.—Small.

Color.—Green-red.

Flowers:

Color.—White.

Number.—Many.

Sexual characteristics.—Triploid, crop is rare.

### FRUIT

Maturity date/location: Mid-season in Dresden, Pilluitz.

Size: Small 1-2 g.

Axial diameter: 1-2 cm.

Skin: Dark red-brown

I claim:

1. A new and distinct variety of Prunus rootstock named ‘Piku 4’ substantially as shown and described.
